(19) United States
(12) Patent Application Publication
(10) Pub. No.: US 2026/0174589 A1
(43) Pub. Date: Jun. 25, 2026

(54) FLUID COLLECTION DEVICES ADJUSTABLE BETWEEN A VACUUM-BASED ORIENTATION AND A GRAVITY-BASED ORIENTATION, AND RELATED SYSTEMS AND METHODS

(71) Applicant: PUREWICK CORPORATION, Covington, GA (US)

(72) Inventor: James David Hughett, Sr., Conyers, GA (US)

(21) Appl. No.: 19/358,647

(22) Filed: Oct. 15, 2025

Related U.S. Application Data
(63) Continuation of application No. 17/996,468, now Pat. No. 12,491,104.
(60) Provisional application No. 63/012,384, filed on Apr. 20, 2020.

(51) Int. Cl. A61F 5/455; A61F 5/453

(52) U.S. Cl. CPC A61F 5/455; A61F 5/453

(57) ABSTRACT

Examples relate to fluid collection devices, and related systems and methods. In an embodiment, a fluid collection device includes a fluid impermeable barrier, one or more openings, and a port. The fluid impermeable barrier at least partially defines a chamber and includes a proximal end region and a distal end region. The one or more openings are positioned to receive fluid discharged from a user and are in fluid communication with the chamber. The port is positioned at the proximal end region of the fluid impermeable barrier. The fluid collection device is adjustable between a vacuum-based orientation and a gravity-based orientation to withdraw fluid through the port of the fluid collection device.

### CROSS-REFERENCE TO RELATED APPLICATIONS

This application is a continuation of U.S. application Ser. No. 17/996,468 filed Oct. 18, 2022, which is a U.S. Nationalization of PCT International Application No. PCT/US2021/027917 filed on 19 Apr. 2021, which claims priority to U.S. Provisional Patent Application No. 63/012,384 filed on Apr. 20, 2020, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to urinary capture devices (e.g., urine or fluid collection devices) adjustable between a vacuum-based orientation and a gravity-based orientation, and related systems and methods. In an embodiment, a fluid collection device includes a fluid impermeable barrier, one or more openings, and a port. The fluid impermeable barrier at least partially defines a chamber and includes a proximal end region and a distal end region. The one or more openings are positioned to receive fluid discharged from a user and in fluid communication with the chamber. The port is positioned at the proximal end region of the fluid impermeable barrier. The fluid collection device is adjustable between a vacuum-based orientation and a gravity-based orientation to withdraw fluid through the port of the fluid collection device.

In an embodiment, a method of collecting urine includes selectively configuring a fluid collection device to one of a gravity-based orientation and a gravity-based orientation. The fluid collection device is selectively adjustable between the vacuum-based orientation and the gravity-based orientation. The method also includes positioning one or more openings on the fluid collection device at least proximate to a urethral opening of the user. The method also includes collecting fluids discharged by the user through the one or more openings and in a chamber of the fluid collection device at least partially defined by a fluid impermeable barrier having a proximal end region and a distal end region. The method also may include drawing the fluids from the chamber through a port positioned at the proximal end region of the fluid impermeable barrier.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a fluid collection system, according to an embodiment.

FIG. 2A is an isometric view of a fluid collection device, according to an embodiment.

FIG. 2B is an exploded view of the fluid collection device of FIG. 2A.

FIG. 2C is a partial rear view of the fluid collection device of FIG. 2A in a vacuum-based orientation for fluid collection with a portion of the fluid collection device removed.

FIG. 2D is a partial rear view of the fluid collection device of FIG. 2A in a gravity-based orientation for fluid collection with a portion of the fluid collection device removed.

FIG. 2E is a cross-sectional view of the fluid collection device of FIG. 2A

FIG. 2F is a top isometric view of the fluid collection device of FIG. 2A in a vacuum-based orientation for fluid collection with a portion of the fluid collection device removed.

FIG. 2G is a top isometric view of the fluid collection device of FIG. 2A in a gravity-based orientation for fluid collection with a portion of the fluid collection device removed.

FIG. 2H is a side isometric view of the fluid collection device of FIG. 2A positioned on a user.

FIG. 3A is a cross-sectional view of a fluid collection device, according to an embodiment.

FIG. 3B is front isometric view of a fluid collection device, according to an embodiment.

FIG. 3C is front isometric view of a fluid collection device, according to an embodiment.

FIG. 3D is front isometric view of a fluid collection device, according to an embodiment.

FIG. 3E is a cross-sectional view of the fluid collection device of FIG. 3D according to an embodiment.

FIG. 4A is a front isometric view of a fluid collection device, according to an embodiment.

FIG. 4B is a front isometric view of a fluid collection device, according to an embodiment.

FIG. 5A is a front isometric view of a fluid collection device, according to an embodiment.

FIG. 5B is a rear isometric view of the fluid collection device of FIG. 5A positioned on a user.

FIG. 5C is an exploded view of the fluid collection device of FIG. 5A in a vacuum-based orientation and configuration for fluid collection.

FIG. 5D is a cross-sectional view of the fluid collection device of FIG. 5A in a vacuum-based orientation and configuration for fluid collection.

FIG. 5E is an exploded view of the fluid collection device of FIG. 5A in a gravity-based orientation and configuration for fluid collection.

FIG. 5F is a cross-sectional view of the fluid collection device of FIG. 5A in a gravity-based orientation and configuration for fluid collection.

FIG. 6A is a front isometric view of a fluid collection device, according to an embodiment.

FIG. 6B is a front isometric view of a fluid collection device, according to an embodiment.

FIG. 6C is a front isometric view of a fluid collection device, according to an embodiment.

FIG. 6D is a front isometric view of a fluid collection device, according to an embodiment.

FIG. 6E is a front isometric view of a fluid collection device, according to an embodiment.

FIG. 7A is a front isometric view of a fluid collection device in a gravity-based orientation and configuration for fluid collection, according to an embodiment.

FIG. 7B is an exploded view of the fluid collection device of FIG. 7A in the gravity-based orientation and configuration for fluid collection.

FIG. 7C is a front isometric view of the fluid collection device of FIG. 7A in a vacuum-based orientation and configuration for fluid collection, according to an embodiment.

FIG. 7D is an exploded view of the fluid collection device of FIG. 7A in the vacuum-based orientation and configuration for fluid collection.

FIG. 7E is a front isometric view of a fluid collection device, according to an embodiment.

FIG. 8A is a front isometric view of a fluid collection device, according to an embodiment.

FIG. 8B is a rear isometric view of the fluid collection device of FIG. 8A.

FIG. 8C is an exploded view of the fluid collection device of FIG. 8A.

FIG. 8D is a cross-sectional view of the fluid collection device of FIG. 8A positioned on a user.

FIG. 9A is a front isometric view of a fluid collection device, according to an embodiment.

FIG. 9B is a cross-sectional view of the fluid collection device of FIG. 9A, according to an embodiment.

FIG. 9C is a front isometric view of a fluid collection device, according to an embodiment.

FIG. 9D is a rear isometric view of the fluid collection device secured to a user.

FIG. 10 is a flow diagram of a method for collecting fluid, according to an embodiment.

### DETAILED DESCRIPTION

Embodiments disclosed herein are fluid collection devices configured to allow a user to switch or otherwise select between vacuum-based and gravity-based urine collection. Embodiments of fluid collection devices described herein also may include various sealing methods that prevent or inhibit urine from leaking from the fluid collection devices and/or as the urine is voided into the fluid collection devices. The fluid collection devices described herein may be used to collect fluids such as urine from females, as well as males having a hidden or buried penis. Many embodiments of fluid collection devices disclosed herein also may include a positioning feature that assists in more accurate placement of the fluid collection device on the user.

Conventional fluid collection devices similar to the fluid collection devices described herein can be limited by the requirement to work with a vacuum, e.g., conventional fluid devices similar to the fluid collection devices described herein will not work with gravity alone. In conventional fluid collection devices, fabric and spun plastic may add significant resistance that degrades fluid capture rate, making gravity-based fluid collection difficult. Many fluid collection devices described herein include an open chamber with or without a fabric cover. This configuration of an open chamber with or without a fabric cover facilitates maximum fluid capture under a gravity-based fluid collection.

In some embodiments, the fluid collection device includes one or more layers of fabric that allows the fluid collection device to operate in either a vacuum-based or gravity-based mode or configuration. The one or more layers of fabric may be interchangeable, such that a fabric layer suitable for gravity-based fluid collection may be interchangeable with a fabric layer suitable for vacuum-based fluid collection. In some embodiments, gravity-based configuration(s) may include additional gravity-based seals to reduce leaks from the fluid collection device if excess fluid is generated.

Fluid collection devices according to one or more embodiments of this disclosure may include a fluid impermeable barrier defining an opening and a port. The port may be positioned at an end of the elongated fluid impermeable barrier and may be configured to connect to a tube or conduit. Various materials and/or configurations may span across at least a portion (or all) of the opening. The opening of the fluid impermeable barrier, including the various materials and/or configurations, may be placed against or proximate to the urethral opening of a female or a male having a hidden or buried penis. Urine discharged from the urethral opening may pass through the opening into a chamber defined by the fluid impermeable barrier, before being withdrawn from the chamber of the fluid impermeable barrier through a tube or conduit secured to the port.

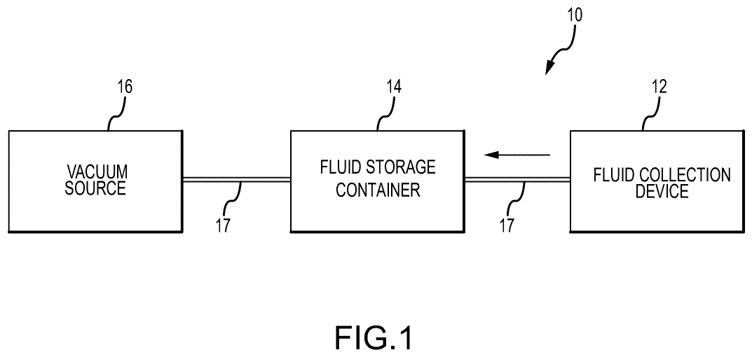

FIG. 1 is a block diagram of a system 10 for fluid collection, according to an embodiment. The system 10 includes a fluid collection device 12, a fluid storage container 14, and a portable vacuum source 16. The fluid collection device 12 may include any of the fluid collection devices described herein. As described in greater detail throughout this document, many fluid collection devices described herein may be adjustable between vacuum-based orientations and gravity-based orientations. Thus, in a vacuum-based orientation, the system 10 may include the vacuum source 16, but in a gravity-based orientation, the vacuum source 16 may be absent. The fluid collection device 12, the fluid storage container 14, and the portable vacuum source 16 may be fluidly coupled to each other via one or more conduits 17 or tubes. The conduit 17 may include any of the conduits or tubes described herein. The fluid collection device 12 may be operably coupled to one or more of the fluid storage container 14 or the portable vacuum source via the conduit 17. Fluid (e.g., urine or other bodily fluids) collected in the fluid collection device 12 may be removed from the fluid collection device 12 via the conduit 17, which protrudes into an interior region of the fluid collection device 12 or has fluid communication with the interior region of the fluid collection device 12. For example, a first open end of the conduit 17 may extend into the fluid collection device 12 to a reservoir therein or may have fluid communication with the interior region of the fluid collection device 12 through a port. The second open end of the conduit 17 may extend into the fluid storage container 14 or the portable vacuum source 16. The suction force may be introduced into the interior region of the fluid collection device 12 via the first open end of the conduit 17 responsive to a suction (e.g., vacuum) or gravitational force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the portable vacuum source 16. Accordingly, the portable vacuum source 16 may apply suction to the fluid collection device 12 via the fluid storage container 14. The suction force may be applied directly via the fluid storage container 14. For example, the second open end of the conduit 17 may be disposed within the portable vacuum source 16. An additional conduit 17 may extend from the portable vacuum source 16 to a point outside of the fluid collection device 12, such as to the fluid storage container 14. In such examples, the portable vacuum source 16 may be disposed between the fluid collection device 12 and the fluid storage container 14. When the vacuum source 17 is absent and the fluid collection device 12 is in a gravity-based orientation, the fluid storage container 14 may be positioned below the fluid collection device 12 and the gravitational force may draw urine from the fluid collection device 12 into the fluid storage container 14.

The fluid collection device 12 may be shaped and sized to be positioned adjacent to a female urethra. The fluid collection member of the fluid collection device 12 may include a fluid impermeable barrier at least partially defining a chamber (e.g., interior region of the fluid collection device member) of the fluid collection device 12. The fluid collection device may include an opening extending therethrough from the external environment. The opening may be positioned on the fluid collection device to be aligned adjacent to a female urethra. The fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e.g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the portable vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection member of the fluid collection device 12. In some embodiments, the portable vacuum source 16 may be powered by one or more of a power cord (e.g., connected to a power socket), one or more batteries, or even manual power (e.g., a hand operated vacuum pump). In examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source 16. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

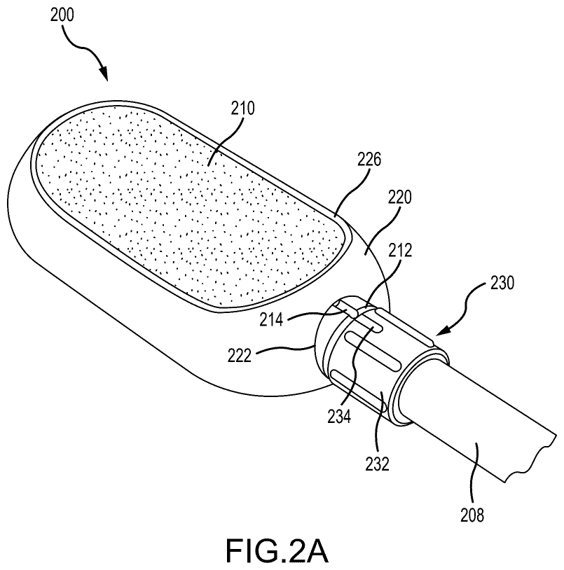

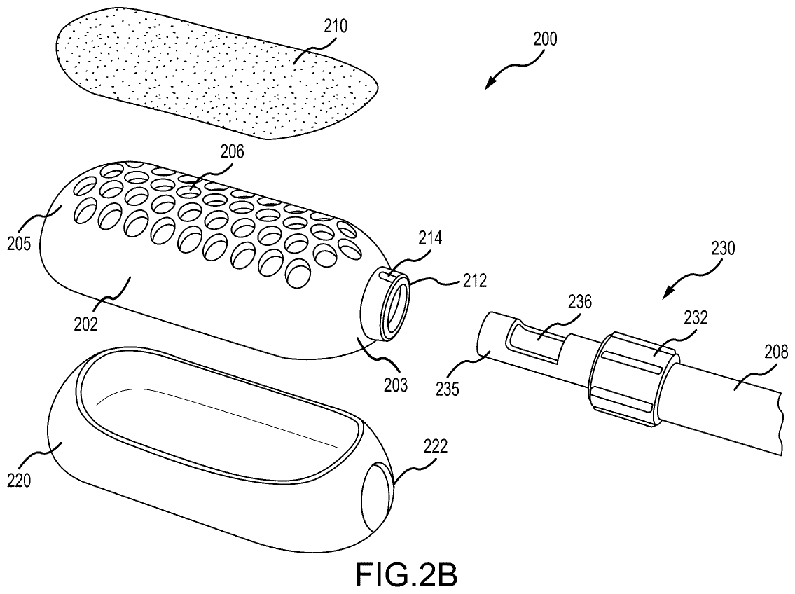

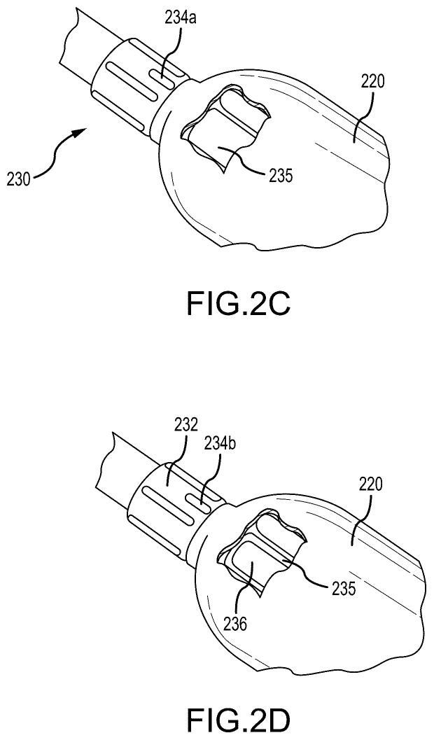

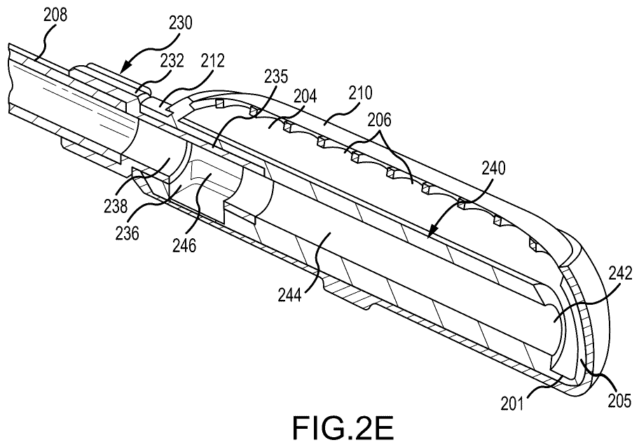

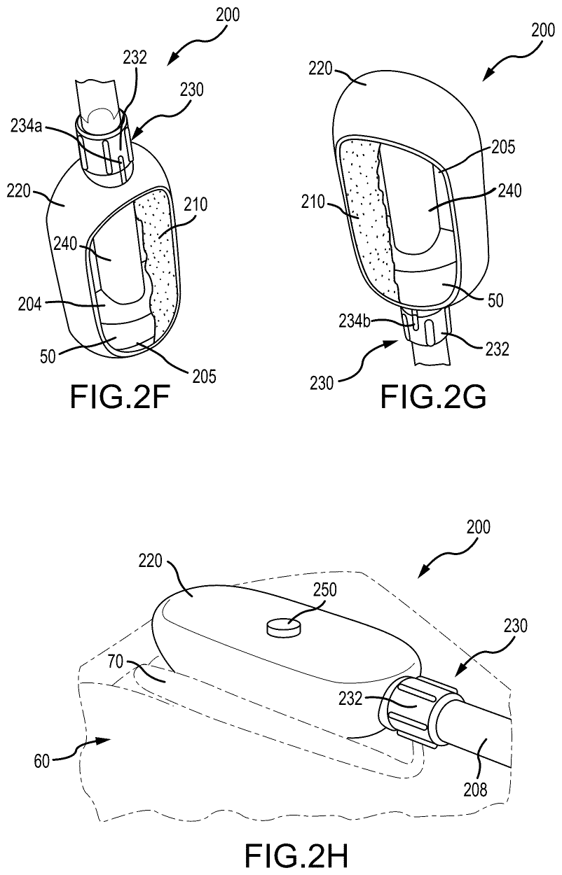

FIGS. 2A-2H illustrate a fluid collection device 200 adjustable for use with either gravity-based or vacuum-based fluid collection, according to an embodiment. FIG. 2A shows an isometric view of an assembled fluid collection device 200, according to an embodiment. FIG. 2B shows an exploded view of the fluid collection device 200. The fluid collection device 200 may include a wicking fabric 210 that pulls moisture away from the user and keeps the area around the urethral opening dry. The fluid collection device 200 also includes a fluid impermeable barrier 202 (e.g., an inner shell) and may include a fluid impermeable shell 222 (e.g., outer shell) that encloses at least a portion of the fluid impermeable barrier 202. The fluid impermeable barrier 202 may at least partially define a chamber 204 (e.g., interior region, shown in FIG. 2E) and one or more (e.g., a plurality of) small openings 206 of a general pattern in fluid communication with the chamber 204 such that the openings 206 allow urine to flow into the chamber 204 when discharged by a user. The fluid impermeable barrier 202 also may include a proximal end region 203, a distal end region 205, and a port 212 positioned at the proximal end region 203 of the fluid impermeable barrier 202. The fluid collection device 200 also includes a flow adapter 230 that allows a user to select and adjust between a gravity-based orientation or configuration and a vacuum-based orientation or configuration for fluid collection.

As illustrated in FIGS. 2E-2G, the internal portion of the fluid impermeable barrier 202 may include a tubular member 240 in the chamber 204 along the back surface 201. This tubular member 240 has one or more gravity inlets 246 or valves and a vacuum inlet 242 that, with the flow adapter 230, allow a user to select and adjust between vacuum-based orientation or configuration and a gravity-based orientation or configuration for fluid collection. The tubular member 240 may extend from the port 212 to the vacuum inlet 242 that is spaced from the distal end region 205 of the fluid impermeable barrier 202 to allow entry of fluid into the tubular member 240 in the vacuum-based orientation. The one or more gravity inlets 246 may be proximate to the proximal end region 203 of the fluid impermeable barrier 202 to facilitate entry of fluid into the tubular member 240 in the gravity-based orientation.

The flow adapter 230 may be rotabably secured or securable to the port 212. As illustrated in FIGS. 2B and 2E, the flow adapter 230 may include a head 232 positioned outside the fluid impermeable barrier 202 and a neck 235 extending through the port 252. The neck 235 may define a passage 238 in fluid communication with a conduit 244 of the tubular member 244. The neck 235 also may define a flow adapter opening 236 positioned to at least partial align with the one or more gravity inlets 246 when the flow adapter 230 and the neck 235 are rotated to a selected position. FIGS. 2C and 2D illustrate a rear view of a portion of the fluid collection device 200 with some of the fluid impermeable barrier 202 removed to allow viewing of the neck 235. The flow adapter 230 is rotatable relative to the fluid impermeable barrier 202 such that rotating the head 232 rotates the neck 235 between the gravity-based orientation and the vacuum-based orientation. In the gravity-based orientation shown in FIGS. 2D and 2E, the passage 238 of the neck 235 and the flow adapter opening 236 are in fluid communication with the chamber 204 through at least the one or more gravity inlets 246 in the tubular member 240. In the vacuum-based orientation shown in FIG. 2C, the neck 235 is rotated such that the tubular member 240 prevents fluid communication between the chamber 204 and the flow adapter opening 236. In the vacuum-based orientation, the tubular member 240 provides fluid communication between the vacuum inlet 242 and the passage 238 of the neck 235 through the conduit 244 of the tubular member 240. In the vacuum-based orientation, the neck 235 is rotated such that the flow adapter opening 236 does not align with the one or more gravity inlets 246 and the neck 235 blocks the gravity inlets 246 from fluid communication with the chamber 204.

FIGS. 2F and 2G illustrate the fluid collection device 200 with a portion of the wicking fabric 210 removed for viewing in the chamber 204. In FIG. 2F, the fluid collection device 200 is in the vacuum-based orientation for fluid collection. In the vacuum-based orientation, urine 50 or other fluids generally fall to the distal end region 205 of the fluid impermeable barrier 202, and the urine 50 is drawn into the inlet 242 and through the tubular member 240 and the flow adapter 230 with a vacuum source. In FIG. 2F, the fluid collection device 200 is in the gravity-based orientation for fluid collection. In the gravity-based orientation, the urine 50 or other fluids generally fall to the proximal end region 203 of the fluid impermeable barrier 202, and the urine 50 is drawn through the one or more gravity inlets 246 and the flow adapter opening 236 into the flow adapter 230 with gravity. The fluid collection device 200 also may include a tube 208 secured or securable to the head 232 of the flow adapter 230 and in fluid communication with the passage 238 of the neck 235 and the conduit 244 of the tubular member 244. The tube 208 also may be in fluid communication with at least one of a vacuum source and/or a fluid storage container.

As noted above, in some embodiments, the fluid collection device 200 includes the fluid impermeable shell 220 (e.g., outer shell). As illustrated in FIGS. 2A-2H, the shell 220 may be secured or securable to the fluid impermeable barrier 202 and enclosing at least a portion of the fluid impermeable barrier 202. The shell 220 may extend at least partially around the port 212. The shell 220 may define an opening positioned such that the plurality of openings 206 in the fluid impermeable barrier 202 are not covered by the shell 220. In some embodiments, the wicking material 210 is positioned at least partially between the shell 220 and the fluid impermeable barrier 202.

At least one of the port 212 or the shell 220 may include an alignment indicator 214 (shown in FIGS. 2A and 2B). Turning to FIGS. 2F and 2G, the head 232 of the flow adapter 230 also may include a gravity alignment 234b and a vacuum alignment indicator 234a. The gravity alignment indicator 234b may indicate the flow adapter 230 is in the gravity-based orientation when the gravity alignment indicator 234b is aligned with the alignment indicator 214 on the port 212 or the shell 220. The vacuum alignment indicator 234a may indicate the flow adapter 230 is in the vacuum-based orientation when the vacuum alignment indicator 234a is aligned with the alignment indicator 214 on the port 212 or the shell 220.

Turning to FIG. 211, the fluid collection device 200 also may include a positioning feature 250 on the shell 220 distal to the opening in the shell 220. The positioning feature 250 may protrude from the shell 220 and may act as a positioning landmark or as a securement location to an undergarment. However, in other embodiments the positioning feature 250 may be omitted. FIG. 2H illustrates the fluid collection device 200 with the plurality of openings 206 positioned over at least the urethra of the user 60. In some embodiments, the fluid collection device 200 is sized and dimensioned to fit between the labia 70 or the vulva of the user 60, and the labia 70 and/or the vulva of the user may assist in retaining the fluid collection device 200 in place over the urethral opening.

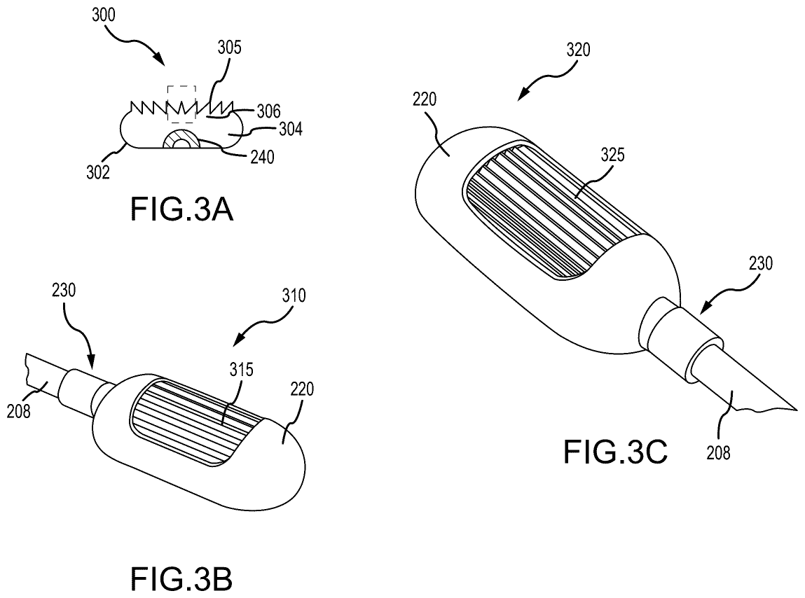

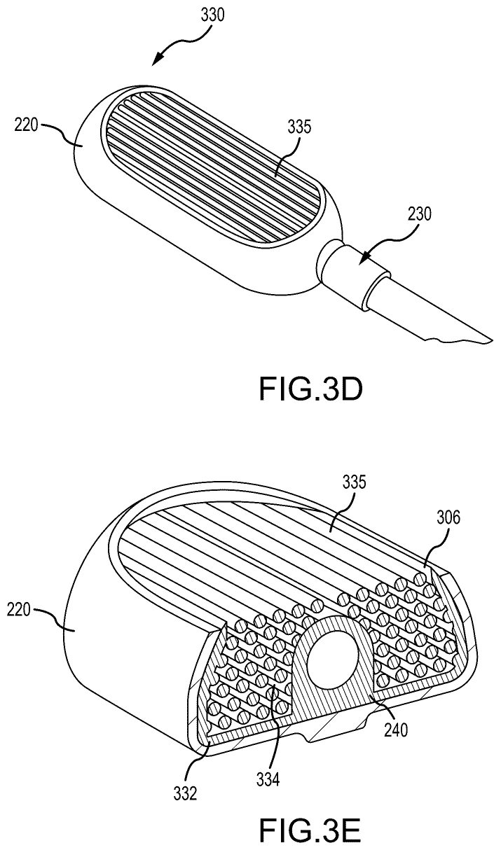

FIGS. 3A-3E illustrate additional embodiments of fluid collection devices that may be used in either vacuum-based or gravity-based fluid collection orientations. Unless otherwise noted, the fluid collection device illustrated in FIGS. 3A-3E may include any aspect of the fluid collection device 200, such as the shell 220, the flow adapter 230, and the tubular member 240. The fluid collection devices illustrated in FIGS. 3A-3E may be configured to collect fluid without a wicking material or other fabric (i.e., the wicking material or fabric may be absent from the fluid collection devices of FIGS. 3A-3E). Turning specifically to FIG. 3A, a fluid collection device 300 may include a plurality of one-way flaps 305 that allow flow in only one direction. More particularly, the fluid collection device 300 may include a fluid impermeable barrier 302 at least partially defining a chamber 304. Air or fluid flow in a direction into the chamber 304 may bias or pull at least some of the plurality of one-way flaps 305 open to allow fluid or air through the openings 306, while fluid flow in a direction out of the chamber 304 may bias or push the plurality of one-way flaps 305 closed. This configuration allows air and/or fluids into the chamber 304 through the openings 306 (such as when urine is discharged and/or a vacuum pulled), but does not allow fluid or air out of the chamber 304 through the openings 306.

Other embodiments may include a string contact surface or a string contact surface combined with a string core. Turning to FIG. 3B, a fluid collection device 310 may include a fluid impermeable barrier (not visible) similar to the fluid impermeable barrier 202, but including a plurality of elongated members 315 (such as strings) extending across one or more openings defined by the fluid impermeable barrier. The fluid impermeable barrier may define a single opening similar to the opening defined by the shell 220 rather than a plurality of openings 206 of the fluid impermeable barrier 202. The plurality of elongated members 315 may include any fluid impermeable or fluid permeable material described herein.

Turning to FIG. 3C, a fluid collection device 320 may include a fluid impermeable barrier (not visible) similar to the fluid impermeable barrier 202, but including a pleated filter 325 extending across one or more openings defined by the fluid impermeable barrier. The pleated filter 325 may allow fluid and air into the chamber of the fluid impermeable barrier, but prevent at least fluid from exiting the chamber through pleated filter 325. The fluid impermeable barrier may define a single opening similar to the opening defined by the shell 220 rather than a plurality of openings 206 of the fluid impermeable barrier 202.

Turning to FIGS. 3D and 3E, a fluid collection device 330 may include a fluid impermeable barrier 332 at least partially defining a chamber 334 and an opening 336. The fluid collection device 330 also may include a plurality of elongated members 335 (such as strings) extending longitudinally across the opening 336 and through the chamber 334. The plurality of elongated members 335 may be arranged into a plurality of rows and/or a plurality of columns in the chamber 334 of the fluid impermeable barrier 332. The plurality of elongated members 335 may include any fluid impermeable or fluid permeable material described herein.

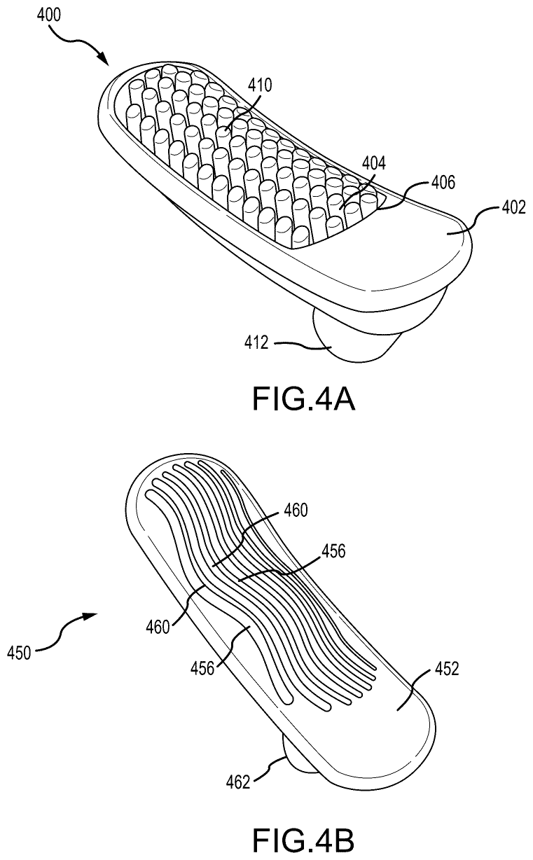

FIGS. 4A and 4B illustrate additional embodiments of fluid collection devices that may be used in either vacuum-based or gravity-based fluid collection orientations. Unless otherwise noted, the fluid collection device illustrated in FIGS. 4A and 4B may include any aspect of the fluid collection device 200, such as the shell 220, the flow adapter 230, and the tubular member 240. In some embodiments, the fluid collection devices illustrated in FIGS. 4A and 4B may operate without the flow adapter 230. The fluid collection devices illustrated in FIGS. 4A and 4B may be configured to collect fluid without a wicking material or other fluid permeable material or fabric (i.e., the wicking material or other fluid permeable material or fabric may be absent from the fluid collection devices of FIGS. 4A and 4B). The fluid collection devices illustrated in FIGS. 4A and 4B may include a one-piece structure configured to secure to a tube for fluid removal. For example, the fluid collection devices illustrated in FIGS. 4A and 4B may by molded as a one-piece structure having a port configured to detachably secure to a tube.

Turning to FIG. 4A, a fluid collection device 400 may include a fluid impermeable barrier 402 at least partially defining a chamber 404 and an opening 406. In some embodiments, the fluid collection barrier 402 includes a front region defining the opening 406, a rear region distal to the front region, and a plurality of tabs 410 extending from the rear region through the chamber 404 towards the opening 406. The plurality of tabs 410 may be substantially cylindrical and may extend to the opening 406 defined by the front region of the fluid impermeable barrier 402. The fluid collection device 400 also includes a port 412 configured to detachably secure to a tube. The port 412 is in fluid communication with the chamber 406 and may be positioned at rear region of the fluid impermeable barrier 402 and/or proximate to an end region of the fluid impermeable barrier 402.

Turning to FIG. 4B, a fluid collection device 450 may include a fluid impermeable barrier 452 at least partially defining a chamber therein and a plurality of openings 456. The plurality of opening 456 may be substantially contoured or wavy and may extend at least partially between the distal end region and the proximal end region of the fluid impermeable barrier 452. The fluid impermeable barrier includes a plurality of contoured or wavy slats 460 or strips extending at least partially between distal end region and the proximal end region. The plurality of openings 456 and the plurality of slats 460 or strips may be arranged to alternate with one another (e.g., a slat is positioned between two openings).

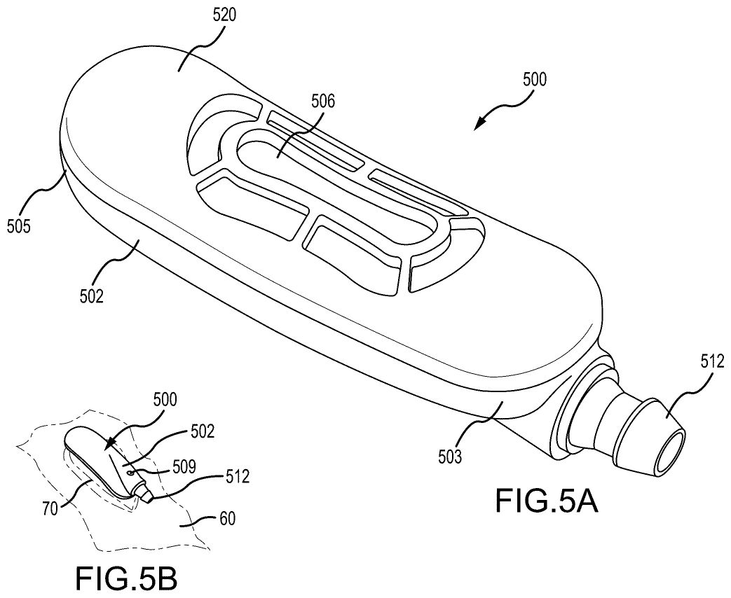

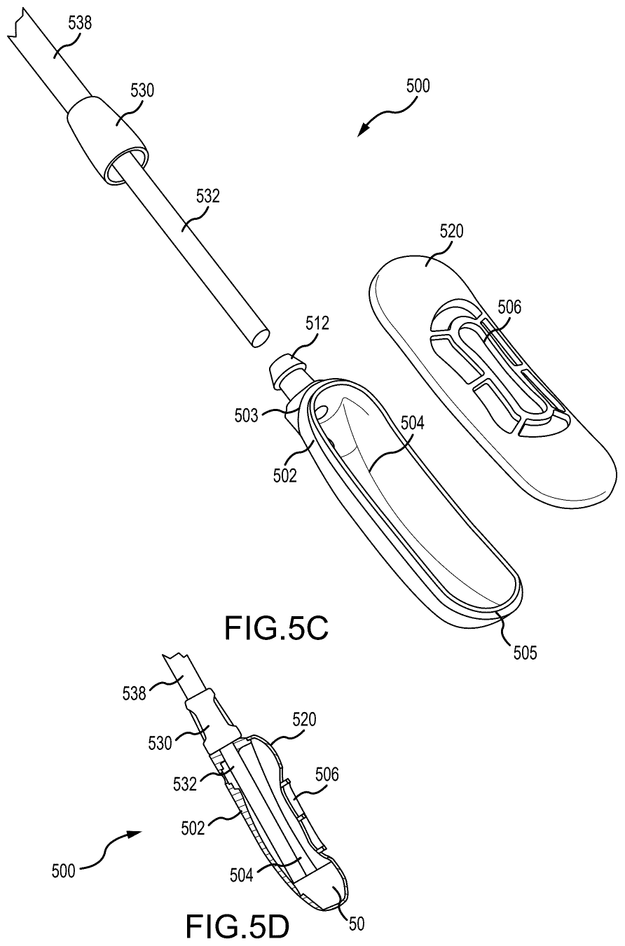

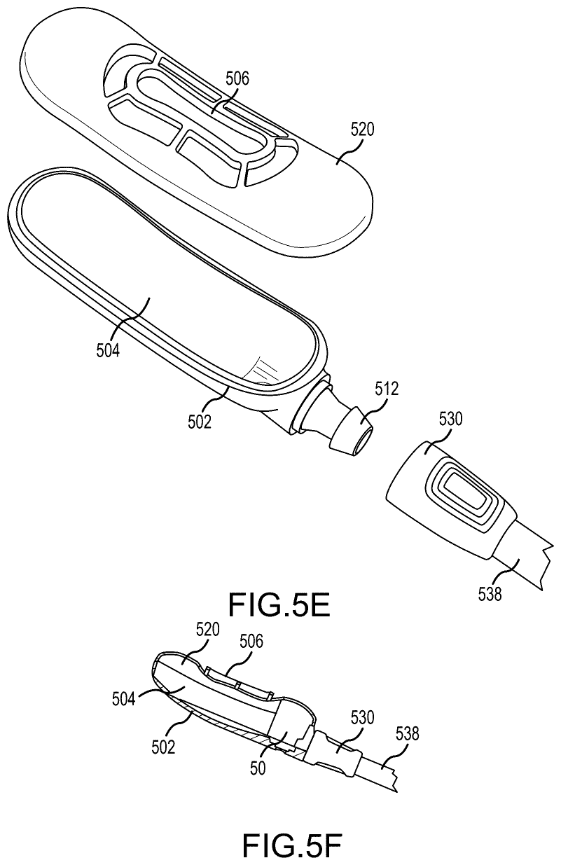

FIGS. 5A-5F illustrate views of a fluid collection device 500, according to various embodiments. The fluid collection device 500 include a connector 530 and an interior tube 532 (shown in FIGS. 5C and 5D) that allows the user to select ahead of time if the device will be used in a vacuum-based or gravity-based orientation for fluid collection, then choose the correct tube(s) or configuration for the selected setting. The fluid collection device 500, then, may be configured and adjusted for dual (gravity-based or vacuum-based) modes of fluid collection. The fluid collection device 500 also may include various sealing and/or securement features or elements. For example, the fluid collection devices may include one or more gels, adhesives, fabric, flexible materials (such as a CPAP mask), or combinations thereof.

FIG. 5A illustrates a front isometric view of the fluid collection device 500, according to an embodiment. The fluid collection device 500 may include a fluid impermeable barrier 502 at least partially defining a chamber 504 (shown in FIG. 5C) and having a proximal end region 503 and a distal end region 505. The fluid collection device 500 may include a fluid impermeable top 520 secured to the fluid impermeable barrier 502. The fluid impermeable top 520 may at least partially define the chamber 504 and may define one or more openings 506 that provide fluid communication with the chamber 504. In some embodiments, the fluid impermeable top 520 includes a soft gel top defining the one or more openings 506 and the fluid impermeable barrier 502 includes a semi-rigid bottom. At least one of the fluid impermeable top or the fluid impermeable barrier 502 includes a port 512 configured to connect to a tube and/or an adapter. The port 512 may be positioned at the proximal end region 503 of the fluid impermeable barrier 502.

FIG. 5B illustrates the fluid collection device 500 positioned over the urethral opening of a user 60. The fluid collection device 500 may be sized and dimensioned to be positioned between and/or held in place by the labia 70 and/or the vulva of the user 60. The fluid collection device 500 also may include a vent 509 configured to allow for air exchange into the chamber 504 while a vacuum is applied to the chamber 604. This air exchange when a vacuum is drawn on the chamber limits suction on tissue to assure comfort for the user 60.

FIG. 5C is an exploded view of the fluid collection device 500 in a vacuum-based orientation or configuration for fluid collection. In some embodiments, the fluid collection device 500 includes the connector 530 and the interior tube 532 that may be used to adapt the fluid collection device 500 for vacuum-based fluid collection. The connector 530 is detachably secured or securable to the port 512. The interior tube 532 may be detachably secured or securable to the connector 530 or the port 512. In some embodiments, the interior tube 532 is detachably secured or securable to the connector 530 and sized to extend through the port 512 into the chamber 502 to extend from the connector 530 towards the distal end region 505 of the fluid impermeable barrier 502. In some embodiments, the interior tube 532 is detachably secured or securable to the port 512 to extend from the port 512 towards the distal end region 505 of the fluid impermeable barrier 502. The fluid collection device also may include a tube 538 secured or securable to the connector 530 opposite to the fluid impermeable barrier 502. The tube 538 is configured to provide fluid communication between the chamber 504 and at least one of a fluid storage container and/or a vacuum source. Whether the interior tube 532 is secured or securable to the port 512 or the connector 530, the interior tube 532 provides fluid communication between the chamber 504 and the tube 538.

FIG. 5D is a cross-sectional view of the fluid collection device 500 with the connector 530 and the interior tube 532 adapted for vacuum-based fluid collection. In this combination and configuration, the interior tube 532 extends through the chamber 504 proximate to a distal end region 505 of the chamber 504 that is distal to the port 512. In the vacuum-based orientation, the urine 50 may collect at the distal end region 505 of the fluid impermeable barrier 502. When a vacuum source initiates a vacuum, the interior tube 532 may draw the urine 50 in the distal end region 505 into the interior tube 532 for removal from the fluid collection device 500. In some embodiments, the connector 530 and interior tube 532 include a tube kit having a straw configuration that extends below the fluid level in the chamber 504, as shown in FIG. 5D.

FIGS. 5E and 5F show the fluid collection device 500 with the connector 530 adapted for gravity-based fluid collection. In this combination, the interior tube 532 is absent or extends only partially into the proximate end region 503 of the chamber 504 that is proximate to the port 512. The straw configuration of the vacuum mode shown in FIG. 5C may be absent, such that fluid 50 in the chamber may flow directly into the port 512 at the proximate end region 503 of the chamber 504. Fluid 50 also may collect at this proximate end region 503 of the chamber 504 due to gravity, and then flow through port 512 and the tube 538 in fluid communication with the port 512 via the connector 530 for withdrawal from the chamber 504.

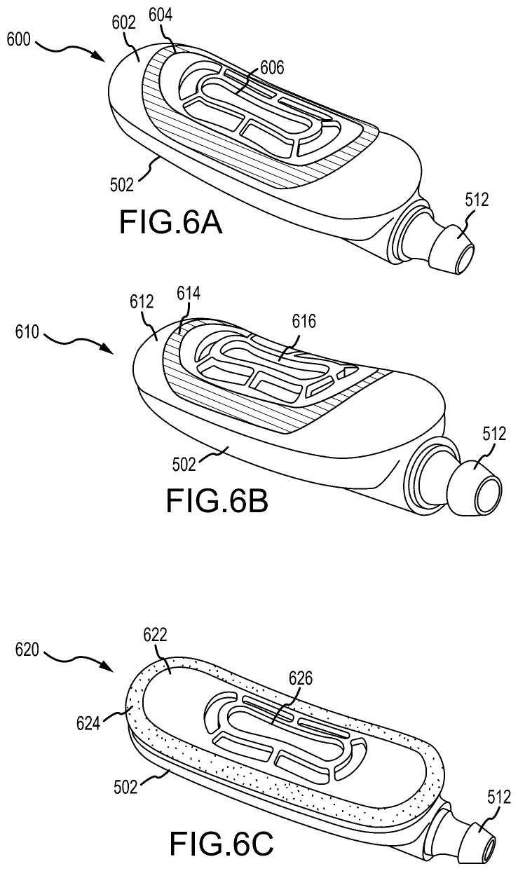

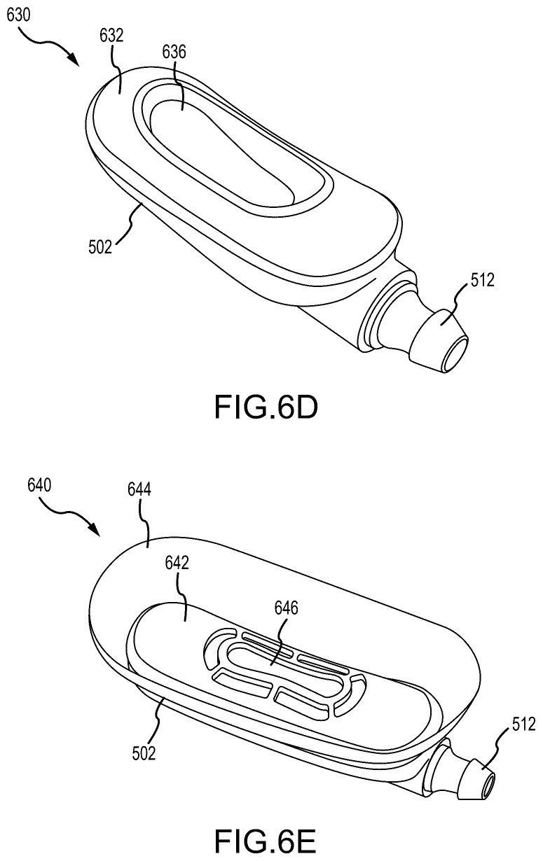

FIGS. 6A-6E illustrate various embodiments of fluid collection devices having different sealing features. Unless otherwise noted, the fluid collection devices of FIGS. 6A-6E may include any aspect of the fluid collection device 600. For example, the fluid collection devices shown in FIGS. 6A-6E may be used with the connector 530 and the interior tube 532 described above for use with either gravity-based or vacuum-based fluid collection. In FIGS. 6A-6E, the fluid collection devices are shown in the gravity-based setting, and the interior tube 532 is absent.

Turning specifically to FIG. 6A, a fluid collection device 600 may include the fluid impermeable barrier 502 and a fluid impermeable top 602 having a gel strip 604. The gel strip 604 may act as a sealing feature and may at least partially (e.g., entirely) surround the one or more openings 606 defined by the fluid impermeable top 602.

Turning specifically to FIG. 6B, a fluid collection device 610 may include the fluid impermeable barrier 502 and a fluid impermeable top 612 having an adhesive ring 614. The adhesive ring 614 may act as a sealing feature and may at least partially (e.g., entirely) surround the one or more openings 616 defined by the fluid impermeable top 612.

Turning specifically to FIG. 6C, a fluid collection device 620 may include the fluid impermeable barrier 502 and a fluid impermeable top 622 having a cotton strip 624 secured thereto or other type of fabric strip. The cotton strip 624 may act as a sealing feature, a comfort feature, and/or a leak prevention feature. The cotton strip 624 may at least partially (e.g., entirely) surround the one or more openings 626 defined by the fluid impermeable top 622. In some embodiments, the cotton strip 624 forms a ring at a periphery or border of the fluid impermeable top 622.

Turning specifically to FIG. 6D, a fluid collection device 630 may include the fluid impermeable barrier 502 and a fluid impermeable top 632 defining an opening 636. The fluid impermeable top 632 and/or the opening 636 may be contoured complementary to the anatomy of the user to act as a sealing feature.

Turning specifically to FIG. 6E, a fluid collection device 640 may include the fluid impermeable barrier 502 and a fluid impermeable top 642 having a flexible flange 644. The flexible flange may act as a sealing feature and may at least partially (e.g., entirely) surround the one or more openings 646 defined by the fluid impermeable top 642. In some embodiments, the flexible flange 644 forms a ring at a periphery or border of the fluid impermeable top 642. The flexible flange 644 may extend outward from the fluid impermeable top 642 away from the fluid impermeable barrier 502. The flexible flange 644 may be secured or securable to at least one of the fluid impermeable barrier 502 and/or the fluid impermeable top 642.

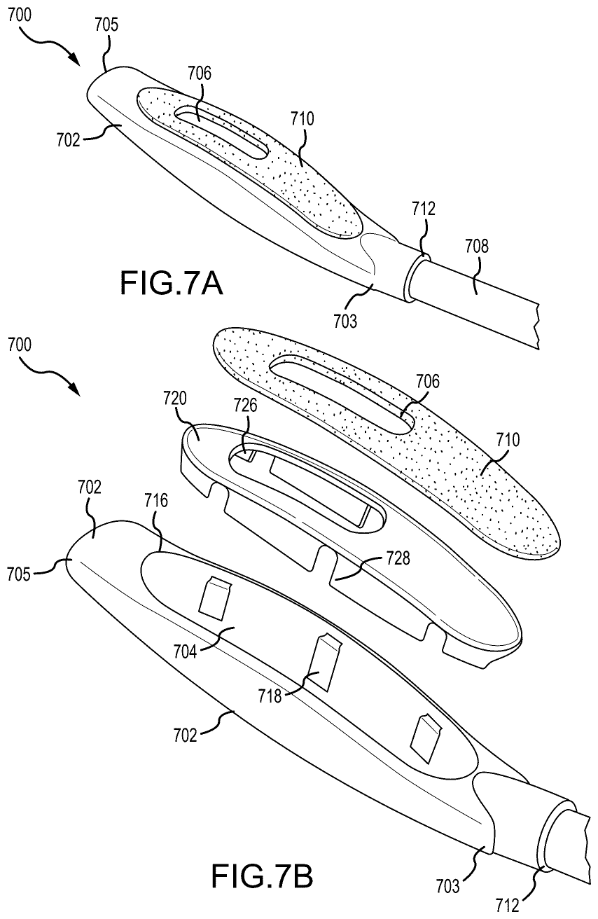

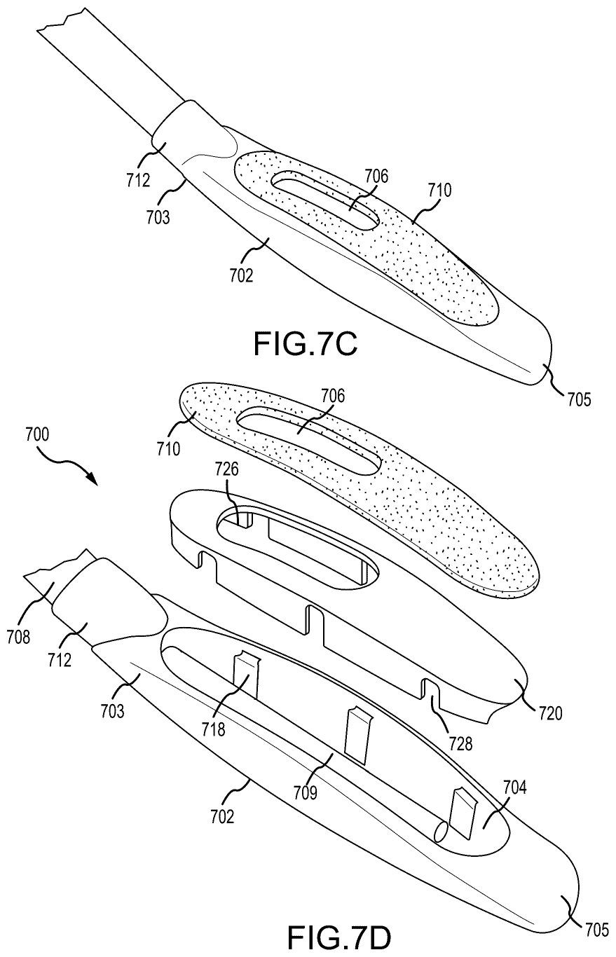

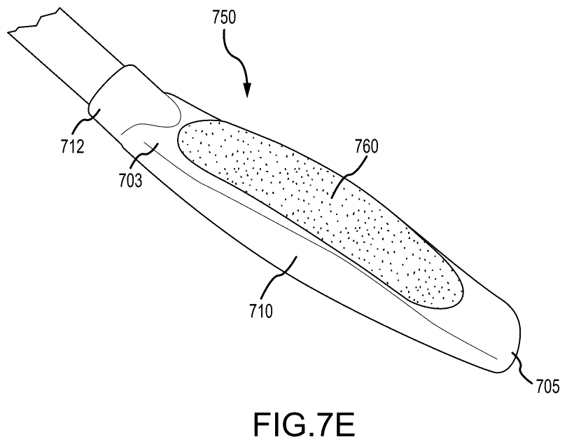

FIGS. 7A-7D illustrate a fluid collection device 700, according to an embodiment. In some embodiments, the fluid collection device 700 may include different attributes to work in both gravity-based and vacuum-based fluid collection settings. The fluid collection device 700 may use many common parts and manufacturing methods to configure both gravity-based and vacuum-based fluid collection devices with minimal change. The fluid collection device 700 may then be packaged as a gravity-based product or a vacuum-based product. In some embodiments, a user may not be able to modify the fluid collection device 700 between gravity-based and fluid-based orientations for fluid collection. In some embodiments, a user may be able to modify or adjust the fluid collection device 700 between gravity-based and fluid-based orientations for fluid collection.

The fluid collection device 700 may include a fluid impermeable barrier 702 (or shell) at least partially defining a chamber 704 and including a proximal end region 703, a distal end region 705, and a port 712 positioned at the proximal end region 703. The port 702 may be configured to connect or secure to a tube 708 in fluid communication with at least one of a fluid storage container and/or a vacuum source. In some embodiments, the fluid collection device 700 includes a fluid impermeable member 720 (or frame) at least partially defining an opening 726 on a top wall thereof and a fluid permeable material 710 covering or secured to the fluid impermeable member 710. The fluid permeable material 710 may at least partially define an opening 706 positioned complementary or aligned with the opening 726 of the fluid impermeable member 710.

Components or parts of the fluid collection device 700 are configured for easy assembly and disassembly to adjust the fluid collection device between the gravity-based orientation and the vacuum-base orientation based on user requirements or preferences. For example, the fluid impermeable barrier 702 may at least partially define an (additional) elongated opening 716 and the fluid impermeable member 720 may detachably secure to the fluid impermeable barrier 702 at least partially in the elongated opening 716. The fluid impermeable barrier 702 may include a plurality of tabs 718 on the interior surface of the fluid impermeable barrier 702 and the fluid impermeable member 720 may include one or more sidewalls having a plurality of slots 728 complementary sized and positioned to align with the plurality of tabs 718 when the fluid impermeable member is inserted into the elongated opening 716. In some embodiments, the opening 726 and the opening 706 may be positioned proximate to an end region of the fluid impermeable member 720 and the fluid permeable material, respectively, and the fluid impermeable member 720 and the fluid permeable material 710 may be rotated relative to the fluid impermeable barrier 702 when securing the fluid permeable member 720 to the fluid impermeable barrier 702.

FIG. 7A shows the fluid collection device 700 in a gravity-based orientation, according to an embodiment, and FIG. 7B shows an exploded view thereof. In the gravity-based orientation, gravity pulls fluid discharged into the chamber 704 of the fluid collection device 700 to the port 712 for removal through the tube 708. FIG. 7C shows the fluid collection device 700 in a vacuum-based orientation, according to an embodiment, and FIG. 7D shows an exploded view thereof. The fluid collection device 700 may include an interior tube 709 that may be used to adapt the fluid collection device 500 for vacuum-based fluid collection. In some embodiments, the interior tube 709 is detachably secured or securable to the port 702 to extend from the port 712 towards the distal end region 705 of the fluid impermeable barrier 702. The tube 708 is configured to provide fluid communication between the chamber 704 and at least one of a fluid storage container and/or a vacuum source. In some embodiments, the port 712 is sized such that the tube 708 may extend through the port 712 and the chamber 704 to position an inlet of the tube 708 proximate to the distal end region 705 of the fluid impermeable barrier 702. Thus, the same fluid collection device 700 is configured to work in either a gravity-based orientation or a vacuum-based orientation by rotating the fluid collection device 700 and/or inserting the tube 708 further into the chamber 704 or withdrawing the tube 708 from the chamber 704.

FIG. 7E illustrates an embodiment of a fluid collection device 750, according to an embodiment. Unless otherwise noted, the fluid collection device 750 may include any aspect of the fluid collection device 700. In some embodiments, the fluid permeable material 760 may cover the opening 726 in the fluid impermeable member 720, and the opening in the fluid permeable member 760 may be absent.

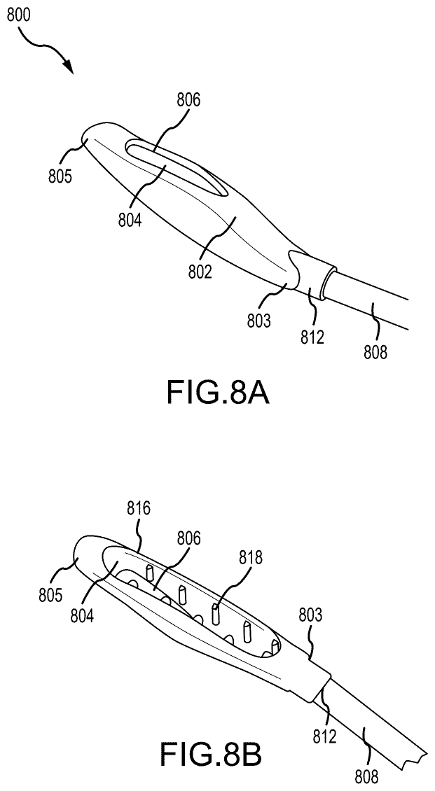

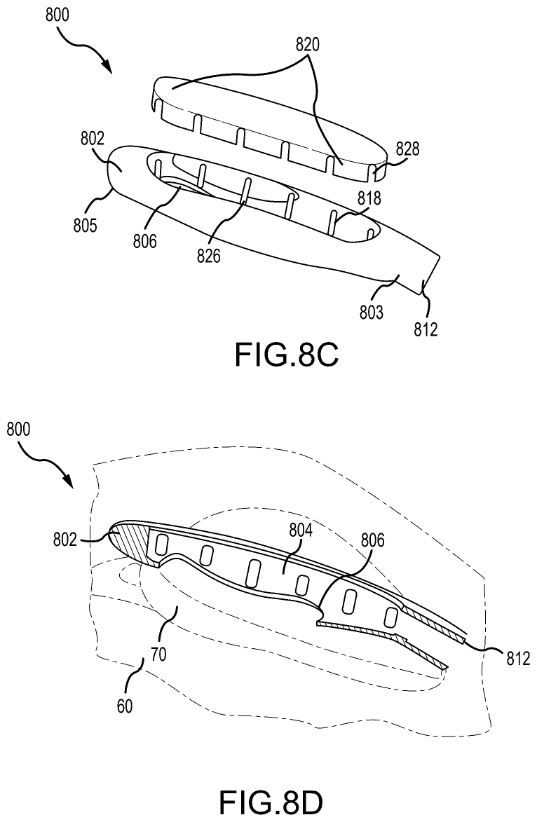

FIGS. 8A-8D illustrate a fluid collection device 800, according to an embodiment. Unless otherwise noted, the fluid collection device 800 may include any aspect of the fluid collection device 700. The fluid collection device may include a fluid impermeable barrier 802 (or outer shell) defining an opening 806 positioned to be placed over the urethra of a user 60. The fluid impermeable barrier 802 may include a proximal end region 803, a distal end region 805, and a port 812 on the proximal end region 803. The fluid impermeable barrier 802 may define an additional elongated opening 816 substantially opposite to the opening 806. The fluid collection device 800 also may include a fluid impermeable member 820 (or an inner shell) configured to be inserted into the elongated opening 816 to detachably secure the fluid impermeable member 820 to the fluid impermeable barrier 802 distal or opposite to the opening 806. In some embodiments, a fluid permeable material or wicking material is absent from the fluid collection device 800.

The fluid impermeable barrier 802 may include a plurality of tabs 818 on the interior surface of the fluid impermeable barrier 802 and the fluid impermeable member 820 may include one or more sidewalls having a plurality of slots 828 complementary sized and positioned to align with the plurality of tabs 818 when the fluid impermeable member is inserted into the elongated opening 816. In some embodiments, the opening 826 and the opening 806 as positioned proximate to an end region of the fluid impermeable member 820 and the fluid permeable material, respectively, and the fluid impermeable member 820 and the fluid permeable material 810 may be rotated relative to the fluid impermeable barrier 802 when securing the fluid permeable member 820 to the fluid impermeable barrier 802.

In some embodiments, the port 812 is sized such that the tube 808 may extend through the port 812 and the chamber 804 to position an inlet of the tube 808 proximate to the distal end region 805 of the fluid impermeable barrier 802. Thus, the same fluid collection device 800 is configured to work in either a gravity-based orientation or a vacuum-based orientation by rotating the fluid collection device 800 and/or inserting the tube 808 further into the chamber 804 or withdrawing the tube 808 from the chamber 804. In some embodiments, the fluid collection device 800 may include an additional interior tube securable to the port to position within the chamber 804. The fluid collection device 800 may be sized and dimensioned to position the opening 806 over the urethral opening of the user 60, with the fluid impermeable barrier 802 positioned at least partially in between the labia 70 and/or the vulva of the user 60.

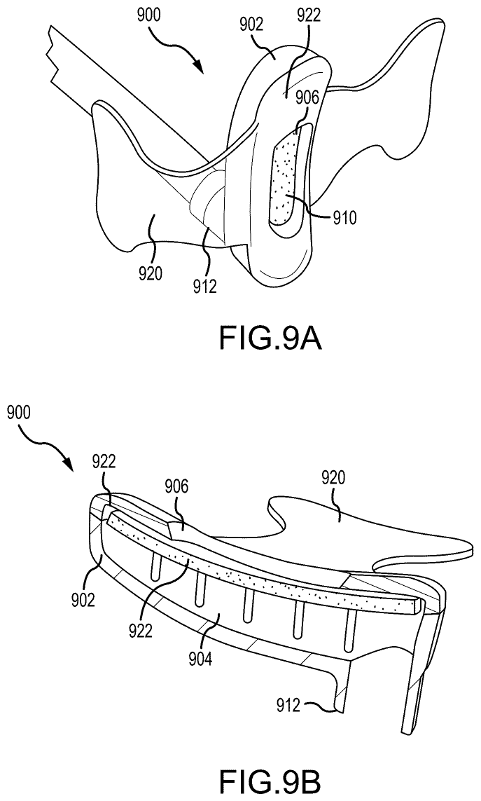

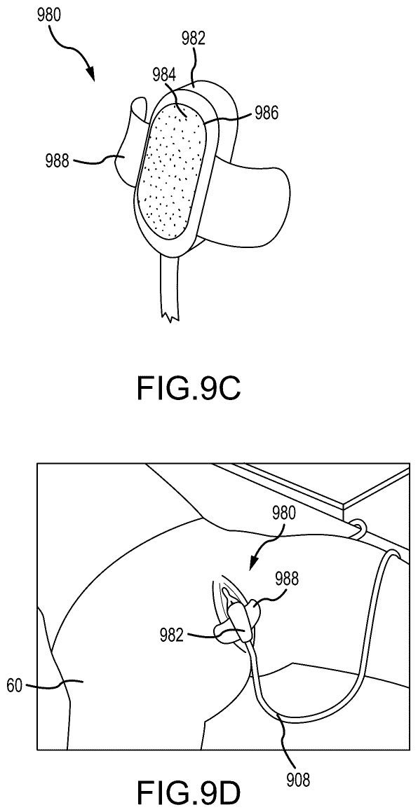

FIG. 9A shows a fluid collection device 900 having an anatomical securement mechanism (such as an adhesive 920) and a thin layer of fabric 910 or other wicking material and/or fluid permeable material, according to an embodiment. FIG. 9B is a cross-sectional view of the fluid collection device 900 of FIG. 9A. In some embodiments, the fabric 910 is absent from the fluid collection device 900. Unless otherwise noted, the fluid collection device 900 may include any aspect of other fluid collection devices described herein. Moreover, the adhesive 920 of the fluid collection device 900 may be included with any fluid collection device described herein.

In some embodiments, the fluid collection device 900 includes a fluid impermeable barrier 902 at least partially defining a chamber 904 and including a proximal end region and a distal end region. The fluid collection device 900 may include a fluid impermeable top 922 secured to the fluid impermeable barrier 902 and at least partially defining an opening 906 in fluid communication with the chamber and positioned to receive fluid discharged from a user. In some embodiments, the fluid impermeable top 922 includes an adhesive region configured to removably secure to the skin of the user. In some embodiments, the fluid impermeable top 922 is absent, and the fluid impermeable barrier 902 at least partially defines the opening 906. The fluid collection device 900 also may include a port 912 configured to secure or connect to a tube and positioned at the proximal end region of the fluid impermeable barrier 902.

The adhesive 920 may be secured or securable to at least one of the fluid impermeable barrier 902 and/or the fluid impermeable top 922. The adhesive 920 may extend laterally away from opposing sides of the fluid collection device 900, thereby providing a winged configuration. The adhesive 920 may be configured to secure to one or more of the skin immediately around the labia and/or the vulva, the skin between the legs of the user, and/or the inner thighs of the user.

In some embodiments, the fluid collection devices described herein may be secured to the user with or without an adhesive. For example, the adhesive 920 may be absent from the fluid collection device 900 and other fluid collection devices described herein, and the fluid collection device 900 and other fluid collection devices described herein may be positioned between the labia or the vulva of the user.

FIGS. 9C and 9D illustrate a fluid collection device 980 having an anatomical securement mechanism (such as adhesive strips 988) and a thin layer of fabric 980 or other wicking material and/or fluid permeable material, according to an embodiment. In some embodiments, the fabric 980 is absent from the fluid collection device 980. Unless otherwise noted, the fluid collection device 980 may include any aspect of other fluid collection devices described herein. Moreover, the adhesive strips 988 of the fluid collection device 980 may be included with any fluid collection device described herein.

In some embodiments, the fluid collection device 980 includes a fluid impermeable barrier 982 at least partially defining a chamber and an opening 986. The fluid collection device 980 also may include a port configured to secure or connect to a tube and positioned at an end region of the fluid impermeable barrier 982. The adhesive strips 988 may be secured or securable to the fluid impermeable barrier 982. The adhesive strips 988 may extend laterally away from opposing sides of the fluid collection impermeable barrier 902. The adhesive strips 988 may be configured to secure to skin immediately around the labia and/or the vulva as shown in FIG. 9D.

The fluid collection devices described herein may be used with either a female user, or a male user having a hidden or buried penis. Fat pads may secure the fluid collection device in place over the urethra opening on the penis, or an adhesive and/or straps may hold the fluid collection device in place over the urethra opening on the penis.

Fluid impermeable barriers or materials described herein may include any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier substantially prevents the fluids from exiting the portions of the chamber that are spaced from the opening. The fluid impermeable barrier is flexible, thereby enabling the fluid collection device to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone. In some embodiments, the fluid impermeable barriers described herein may be air permeable. In these and other embodiments, the fluid impermeable barrier may be formed of a hydrophobic material that defines a plurality of pores.

In embodiments including wicking fabric or other fluid permeable materials, the wicking fabric or material described herein may be configured to wick any fluid away from the anatomy, thereby preventing the fluid from escaping the chamber. The wicking fabric or other fluid permeable materials also may wick the fluid generally towards an interior of the chamber. The wicking fabric or other fluid permeable materials described herein may include any material that may wick the fluid. The permeable properties referred to herein may be wicking, capillary action, diffusion, or other similar properties or processes, and are referred to herein as “permeable” and/or “wicking.” Such “wicking” or other physical properties may exclude absorption into the wicking material or other fluid permeable materials, such as not include adsorption of the bodily fluids into the wicking material or other fluid permeable materials. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the wicking material or other fluid permeable materials (e.g., absorbency), such as less than about 30 wt % of the dry weight of the wicking material or other fluid permeable materials, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the wicking material or other fluid permeable materials. In an embodiment, the wicking material or other fluid permeable materials may include at least one absorbent or adsorbent material.

The wicking material or other fluid permeable materials can include a one-way fluid movement fabric. As such, the wicking material or other fluid permeable materials can remove fluid from the area around the penis, thereby leaving the area and urethra dry. The wicking material or other fluid permeable materials can enable the fluid to flow generally towards the tube within the chamber. The wicking material or other fluid permeable materials can include a porous or fibrous material, such as hydrophilic polyolefin. In some embodiments, the wicking material or other fluid permeable materials consists of or consists essentially of a porous or fibrous material, such as hydrophilic polyolefin. Examples of polyolefin that can be used in the wicking material or other fluid permeable materials include, but are not limited to, polyethylene, polypropylene, polyisobutylene, ethylene propylene rubber, ethylene propylene diene monomer, or combinations thereof. Moreover, the wicking material or other fluid permeable materials can be manufactured according to various manufacturing methods, such as molding, extrusion, or sintering. The wicking material or other fluid permeable materials can include varying densities or dimensions.

In some embodiments, the wicking material or other fluid permeable materials can include two or more layers of fluid permeable materials. For example, the wicking material or other fluid permeable materials can include a fluid permeable membrane covering or wrapped around a fluid permeable support, with both the fluid permeable membrane and the fluid permeable support being disposed in the chamber. The fluid permeable membrane can cover or extend across at least a portion (e.g., all) of at least the side of the fluid permeable support facing the penis of the user. The fluid permeable membrane and the fluid permeable support can be configured to wick any fluid away from the opening, thereby preventing the fluid from escaping the chamber and promoting removal of the fluid through the tube. The permeable properties referred to herein can be wicking, capillary action, diffusion, or other similar properties or processes, and are referred to herein as “permeable” and/or “wicking.”

The fluid permeable membrane and the fluid permeable support also can wick and/or allow transport of the fluid generally towards an interior of the chamber and/or the tube. The fluid permeable membrane can include any material that can wick the fluid. For example, the fluid permeable membrane can include fabric, such as a gauze (e.g., a silk, linen, polymer based materials such as polyester, or cotton gauze), nylon (such as a spun nylon fibers), another soft fabric (e.g., jersey knit fabric or the like), or another smooth fabric (e.g., rayon, satin, or the like). Forming the fluid permeable membrane from gauze, soft fabric, and/or smooth fabric can reduce chaffing caused by the urine collection device 200. Other embodiments of fluid permeable membranes and fluid permeable supports are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid collection devices may include a fluid permeable support including a porous spun nylon fiber structure and a fluid permeable wicking membrane including gauze at least partially enclosing the spun nylon fiber structure. For example, the fluid collection devices may include a gauze or other wicking fabric positioned to contact the skin of the user. In some embodiments, the gauze or other wicking fabric is wrapped around a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

In an embodiment, one or more components of the fluid collection device may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device (e.g., impermeable barrier, conduit, etc.) may include an odor blocking or absorbing material such as a cyclodextrine containing material or a thermoplastic elastomer (TPE) polymer.

Adhesives described herein may include any suitable adhesives, such as a hydrogel layer and those disclosed in U.S. Patent Application Publication No. 2017/0189225, the disclosure of which is incorporated herein by reference in its entirety.

In any of the embodiments disclosed herein, the conduits (or tubes) may include or be operably coupled to a flow meter (not shown) to measure the flow of fluids therein, one or more securement devices (e.g., a StatLock securement device, not shown) or fittings to secure the conduit to one or more components of the systems or devices disclosed herein (e.g., portable vacuum source or fluid storage container), or one or more valves to control the flow of fluids in the systems and devices herein. In an embodiment, at least one of portion of the conduit of the fluid collection devices or systems herein may be formed of an at least partially opaque material which may obscure the fluids that are present therein. For example, a first section of the conduit disclosed herein may be formed of an opaque material or translucent material while a second section of the conduit may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

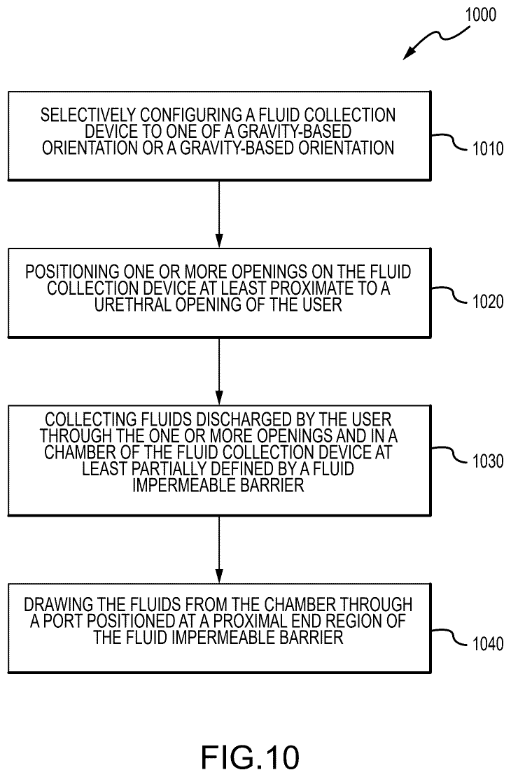

FIG. 10 is a flow diagram of a method 1000 for collecting fluid from a user, according to an embodiment. The method includes an act 1010 of selectively configuring a fluid collection device to one of a gravity-based orientation and a gravity-based orientation. The fluid collection device may be selectively adjustable between the vacuum-based orientation and the gravity-based orientation. The method also includes an act 1020 of positioning one or more openings on the fluid collection device at least proximate to a urethral opening of the user. The method also includes an act 1030 of collecting fluids discharged by the user through the one or more openings and in a chamber of the fluid collection device at least partially defined by a fluid impermeable barrier having a proximal end region and a distal end region. The method also includes an act 1040 of drawing the fluids from the chamber through a port positioned at the proximal end region of the fluid impermeable barrier.

In some embodiments, the act 1010 of selectively configuring a fluid collection device to one of a gravity-based orientation and a vacuum-based orientation may include selectively configuring the fluid collection device to the gravity-based orientation by rotating a head of a flow adapter rotatably secured to the port to align a flow adapter opening defined by a neck of the flow adapter with one or more gravity inlets on a tubular member proximate to the proximal end region. The tubular member may extend from the port to a vacuum inlet spaced from the distal end region. In these and other embodiments, the act 1040 of drawing the fluids from the chamber through a port positioned at the proximal end region of the fluid impermeable barrier may include drawing the fluids from the chamber through the one or more gravity inlets, the flow adapter opening, and the port with gravity. The method 1000 also may include aligning a gravity alignment indicator on the head of the flow adapter with a first alignment indicator on at least one of the fluid impermeable barrier or a shell secured to the fluid impermeable barrier.

In some embodiments, the act 1010 of selectively configuring a fluid collection device to one of a gravity-based orientation and a vacuum-based orientation may include selectively configuring the fluid collection device to the vacuum-based orientation by rotating a head and neck of a flow adapter rotatably secured to the port to close one or more gravity inlets on a tubular member proximate to the proximal end region with the neck. The tubular member may extend from the port to a vacuum inlet spaced from the distal end region. In these and other embodiments, the act 1040 of drawing the fluids from the chamber through a port positioned at the proximal end region of the fluid impermeable barrier may include drawing the fluids from the chamber through the vacuum inlet and the port with gravity with a vacuum source. The method 1000 also may include an act of aligning a vacuum alignment indicator on the head of the flow adapter with a first alignment indicator on at least one of the fluid impermeable barrier or a shell secured to the fluid impermeable barrier.

In some embodiments, the act 1030 of collecting fluids discharged by the user through the one or more openings and a chamber of the fluid collection device may include collecting fluids discharged by the user through a wicking material secured to the fluid collection device and covering the one or more openings, through the one or more openings, and into the chamber of the fluid collection device. In some embodiments, the act 1030 of collecting fluids discharged by the user through the one or more openings and a chamber of the fluid collection device may include collecting fluids discharged by the user through a plurality of one way flaps extending across the one or more openings, through the one or more openings, and into the chamber of the fluid collection device, the plurality of one way flaps preventing the fluid from exiting chamber. In some embodiments, the act 1030 of collecting fluids discharged by the user through the one or more openings and a chamber of the fluid collection device may include collecting fluids discharged by the user in between a plurality of strings extending across the one or more openings and into the chamber of the fluid collection device. In some embodiments, the act 1030 of collecting fluids discharged by the user through the one or more openings and a chamber of the fluid collection device may include collecting fluids discharged by the user through a pleated filter to the fluid collection device and covering the one or more openings, through the one or more openings, and into the chamber of the fluid collection device. In some embodiments, the act 1030 of collecting fluids discharged by the user through the one or more openings and a chamber of the fluid collection device may include collecting fluids discharged by the user through a wicking material secured to the fluid collection device and covering the one or more openings, through the one or more openings, and into the chamber of the fluid collection device.

In some embodiments, the act 1010 of selectively configuring a fluid collection device to one of a gravity-based orientation and a vacuum-based orientation may include selectively configuring the fluid collection device to the gravity-based orientation by securing a connector to the port, the fluid collection device including a fluid impermeable top secured to the fluid impermeable barrier at least partially defining the chamber and the one or more openings. In these and other embodiments, the act 1040 of drawing the fluids from the chamber through a port positioned at the proximal end region of the fluid impermeable barrier may include drawing the fluids from the chamber through the port and the connector with gravity.

In some embodiments, the act 1010 of selectively configuring a fluid collection device to one of a gravity-based orientation and a vacuum-based orientation may include selectively configuring the fluid collection device to the vacuum-based orientation by securing an interior tube to a connector, inserting the interior tube through the port at least partially into the chamber, and securing the connector to the port. In these and other embodiments, the act 1040 of drawing the fluids from the chamber through a port positioned at the proximal end region of the fluid impermeable barrier may include drawing the fluids from the chamber through the interior tube, the port, and the connector into tube with a vacuum source.

In some embodiments, the act 1010 of selectively configuring a fluid collection device to one of a gravity-based orientation and a vacuum-based orientation may include selectively configuring the fluid collection device to the gravity-based orientation by inserting a fluid impermeable member defining the one or more openings into an elongated additional opening of the fluid impermeable barrier with the one or more openings proximate to the proximal end region of the fluid impermeable barrier. In these and other embodiments, the act 1040 of drawing the fluids from the chamber through a port positioned at the proximal end region of the fluid impermeable barrier may include drawing the fluids from the chamber through the port with gravity.

In some embodiments, the act 1010 of selectively configuring a fluid collection device to one of a gravity-based orientation and a vacuum-based orientation may include selectively configuring the fluid collection device to the vacuum-based orientation by inserting an interior tube through the port at least partially into the chamber and inserting a fluid impermeable member defining the one or more openings into an elongated additional opening of the fluid impermeable barrier with the one or more openings proximate to the distal end region of the fluid impermeable barrier. In these and other embodiments, the act 1040 of drawing the fluids from the chamber through a port positioned at the proximal end region of the fluid impermeable barrier may include drawing the fluids from the chamber through the interior tube and the port with a vacuum source.

In some embodiments, the method 1000 may include an act of adhering the fluid collection device to the user with an adhesive secured to the fluid impermeable barrier. In some embodiments, the act 1020 of positioning one or more openings on the fluid collection device at least proximate to a urethral opening of the user may include positioning the fluid collection device between the labia of the user with the one or more openings on the fluid collection device at least proximate to the urethral opening of the user.

The acts of the method 1000 described above are for illustrative purposes. For example, the acts of the method 1000 can be performed in different orders, split into multiple acts, modified, supplemented, or combined. In an embodiment, one or more of the act of the method 1000 can be omitted from the method 1000. Any of the acts of the method 1000 can include using any of the portable urine collection systems disclosed herein.

As used herein, the term “about” or “substantially” refers to an allowable variance of the term modified by “about” by +10% or +5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting. Features from any of the disclosed embodiments may be used in combination with one another, without limitation. In addition, other features and advantages of the present disclosure will become apparent to those of ordinary skill in the art through consideration of the detailed description and the accompanying drawings.

1. A fluid collection device, comprising:
a fluid impermeable barrier at least partially defining a chamber and including a proximal end region and a distal end region;
one or more openings for receiving fluid discharged from a user and in fluid communication with the chamber; and
a port positioned at the proximal end region of the fluid impermeable barrier;
a tube and in fluid communication with the chamber through the port;
a rotatable member secured to the proximal end region of the fluid impermeable barrier and including a first inlet positioned within the chamber, the rotatable member being rotatable between (1) a first position in which the first inlet provides fluid communication between the chamber and the tube and (2) a second position in which fluid communication between the chamber and the tube through the first inlet is absent; and
a conduit positioned within the chamber and in fluid communication with the tube, the conduit including a second inlet positioned distal to the first inlet.

2. The fluid collection device of claim 1, wherein the rotatable member includes a neck at least partially positioned within the chamber and defining the first inlet and a distal inlet in fluid communication with the conduit.

3. The fluid collection device of claim 2, wherein the rotatable member includes a head positioned outside the chamber, and wherein rotation of the head rotates the neck in the chamber between the first position and the second position.

4. The fluid collection device of claim 3, wherein the tube is secured to the head proximal of the neck.

5. The fluid collection device of claim 1, further comprising a wicking material extending across the one or more openings.

6. The fluid collection device of claim 5, further comprising a fluid impermeable shell at least partially encasing the fluid impermeable barrier, the fluid impermeable shell defining an elongated opening and an aperture.

7. The fluid collection device of claim 6, wherein the fluid impermeable barrier is positioned at least partially within the fluid impermeable shell with the port extending through the aperture in the fluid impermeable shell, and wherein the one or more openings of the fluid impermeable barrier include a plurality of openings not covered by the fluid impermeable shell and with the wicking material extending across the plurality of openings.

8. A fluid collection device, comprising:
one or more fluid impermeable members having an elongated configuration, at least one fluid impermeable member of the one or more fluid impermeable members having a proximal end region and a distal end region, the at least one fluid impermeable member defining an opening that is elongated between the proximal end region and the distal end region and an aperture at the proximal end region;
a fluid permeable material extending across the opening of the at least one fluid impermeable barrier;
a chamber positioned within the one or more fluid impermeable members;
a tube in fluid communication with the chamber through the aperture; and
an adjustable member secured to the proximal end region of the at least one fluid impermeable barrier and including a first inlet positioned within the chamber, the adjustable member being adjustable between (1) a first position in which the first inlet provides fluid communication between the chamber and the tube and (2) a second position in which fluid communication between the chamber and the tube through the first inlet is absent.

9. The fluid collection device of claim 8, further comprising a conduit positioned within the chamber and in fluid communication with the tube, the conduit including a second inlet positioned distal to the first inlet.

10. The fluid collection device of claim 9, wherein the adjustable member includes a neck at least partially positioned within the chamber and defining the first inlet and a distal inlet in fluid communication with the conduit.

11. The fluid collection device of claim 10, wherein the adjustable member includes a head positioned outside the chamber at least proximate to the aperture, and wherein rotation of the head rotates the neck in the chamber between the first position and the second position.

12. The fluid collection device of claim 11, wherein the tube is secured to the head proximal of the neck.

13. The fluid collection device of claim 9, wherein the one or more fluid impermeable members include:
a fluid impermeable shell having the proximal end region and the distal end region, the fluid impermeable shell defining the opening that is elongated between the proximal end region and the distal end region and the aperture at the proximal end region; and
a fluid impermeable barrier defining a plurality of openings and the chamber, the fluid impermeable barrier including a port extending through the aperture in the fluid impermeable shell;
wherein the fluid impermeable shell at least partially encases the fluid impermeable barrier.

14. The fluid collection device of claim 13, wherein the fluid impermeable barrier includes a conduit positioned within the chamber and in fluid communication with the tube, the conduit including a second inlet positioned distal to the first inlet.

15. The fluid collection device of claim 14, wherein the plurality of openings of the fluid impermeable barrier are not covered by the fluid impermeable shell and the wicking material extends across the plurality of openings.

16. A method of collecting fluid from a user, the method comprising:
providing a fluid collection device having:
one or more fluid impermeable members having an elongated configuration, at least one fluid impermeable member of the one or more fluid impermeable members having a proximal end region and a distal end region, the at least one fluid impermeable member defining an opening that is elongated between the proximal end region and the distal end region and an aperture at the proximal end region;
a fluid permeable material extending across the opening of the at least one fluid impermeable barrier;
a chamber positioned within the one or more fluid impermeable members;
a tube in fluid communication with the chamber through the aperture; and
an adjustable member secured to the proximal end region of the at least one fluid impermeable barrier and including a first inlet positioned within the chamber, the adjustable member being adjustable between (1) a first position in which the first inlet provides fluid communication between the chamber and the tube and (2) a second position in which fluid communication between the chamber and the tube through the first inlet is absent;
selectively adjusting the adjustable member from the first position to the second position;
positioning the fluid permeable material at least proximate to a urethral opening of the user;
collecting fluids discharged by the user through the elongated opening and the fluid permeable material into the chamber; and
drawing fluids from the chamber through tube.

17. The method of claim 16, further comprising a conduit positioned within the chamber and in fluid communication with the tube, the conduit including a second inlet positioned distal to the first inlet.

18. The method of claim 17, wherein:
the adjustable member includes:
a neck at least partially positioned within the chamber and defining the first inlet and a distal inlet in fluid communication with the conduit; and
a head positioned outside the chamber at least proximate to the aperture; and
selectively adjusting the adjustable member from the first position to the second position includes rotating the head to rotate the neck in the chamber between the first position and the second position.

19. The method of claim 16, wherein the one or more fluid impermeable members include:
a fluid impermeable shell having the proximal end region and the distal end region, the fluid impermeable shell defining the opening that is elongated between the proximal end region and the distal end region and the aperture at the proximal end region; and
a fluid impermeable barrier defining a plurality of openings and the chamber, the fluid impermeable barrier including a port extending through the aperture in the fluid impermeable shell;
wherein the fluid impermeable shell at least partially encases the fluid impermeable barrier.

20. The method of claim 19, wherein:
the fluid impermeable barrier includes a conduit positioned within the chamber and in fluid communication with the tube, the conduit including a second inlet positioned distal to the first inlet; and
the plurality of openings of the fluid impermeable barrier are not covered by the fluid impermeable shell and the wicking material extends across the plurality of openings.
